(19) United States
(12) Patent Application Publication
(10) Pub. No.: US 2017/0119678 A1
(43) Pub. Date: May 4, 2017

(54) EXTENDED RELEASE COMPOSITIONS OF CARVEDILOL PHOSPHATE

(71) Applicant: HETERO RESEARCH FOUNDATION, HYDERABAD, ANDHRA PRADESH (IN)

(72) Inventors: BANDI PARTHASARADHI REDDY, ANDHRA PRADESH (IN); PODILI KHADGAPATHI, HYDERABAD, ANDHRA PRADESH (IN); SAHOO BIJAYANANDA, HYDERABAD, ANDHRA PRADESH (IN)

(21) Appl. No.: 15/320,080

(22) PCT Filed: Jun. 8, 2015
(86) PCT No.: PCT/IN2015/000233

§ 371 Date: Dec. 19, 2016

(30) Foreign Application Priority Data
Jun. 23, 2014 (IN) 3040/CHE/2014

(51) Int. Cl. A61K 9/48; A61K 9/20; A61K 31/404

(52) U.S. Cl. CPC A61K 9/4808; A61K 31/404; A61K 9/2031; A61K 9/2027

(57) ABSTRACT

The present invention relates to solid oral dosage forms of carvedilol phosphate. More specifically, the present invention relates to extended release compositions of carvedilol phosphate and process for their preparation.

### PRIORITY

This patent application claims priority to Indian patent application number 3040/CHE/2014, filed on Jun. 23, 2014, the contents of which are incorporated by reference herein in their entirety.

### FIELD OF THE INVENTION

The present invention relates to solid oral dosage forms of carvedilol or a pharmaceutically acceptable salt thereof. More particularly, the present invention relates to extended release compositions of carvedilol phosphate.

### BACKGROUND OF THE INVENTION

Carvedilol phosphate, a nonselective β-adrenergic blocking agent with al-blocking activity is chemically described as (2RS)-1(9H-Carbazol-4-yloxy)-3-[[2-(2-methoxyphenoxy) ethyl]amino]propan-2-olphosphate salt (1:1) hemihydrate. It is a racemic mixture with the following structure:

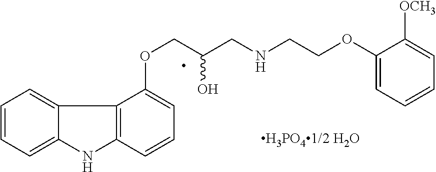 [SMILES: COc1ccccc1OCCNCC(O)COc1cccc2[nH]c3ccccc3c12]

In the United States, Carvedilol phosphate is available as extended release capsules containing 10 mg, 20 mg, 40 mg, 80 mg carvedilol phosphate with trade name Coreg®CR by SB Pharmco.

U.S. Pat. Nos. 4,503,067 and 5,071,868, assigned to Boehringer, disclose carvedilol and its salts.

U.S. Pat. No. 6,022,562, assigned to Flamel Technologies, disclose reservoir type microcapsules comprising drug coated with a coating comprising film forming polymer, nitrogen-containing polymer, plasticizer, surface-active and/or lubricating agent.

U.S. Pat. No. 8,101,209, assigned to Flamel Technologies, claims delayed and controlled release microparticles coated with at least one hydrophilic polymer A carrying groups that are ionized at neutral pH, and at least one hydrophobic compound B.

U.S. Patent Application Publication No. 2005/0175695, assigned to GlaxoSmithKline, claims microparticle composition comprising a mixture of rapidly releasing microparticles and at least two types of controlled release microparticles.

U.S. Patent Application Publication No. 2010/0021549, assigned to Flamel Technologies, describes reservoir-type microparticles of carvedilol non-covalently combined, at least in part, with nanoparticles of at least one polyamino acid polymer, abbreviated to “POM”.

U.S. Patent Application Publication No. 2009/0220611, assigned to Flamel Technologies, describes microparticles comprising drug, coating film A and different coating film B comprising hydrophilic polymer bearing group that is ionized at neutral pH with two different triggering mechanisms of drug.

U.S. Patent Application Publication No. 2012/0245212, assigned to TSH Biopharm, disclose controlled release formulation comprising two or more subunits wherein at least one of said subunits is an immediate release carvedilol subunit and at least one of said subunits is a slow release carvedilol subunit.

There remains a need to develop alternative extended release compositions of carvedilol phosphate using simple techniques. Accordingly, inventors of the present invention have developed compositions of carvedilol phosphate that were found to be comparable with marketed Coreg CR® capsules.

### SUMMARY OF THE INVENTION

The present invention relates to solid oral dosage forms comprising carvedilol phosphate and one or more pharmaceutically acceptable excipients.

The present invention particularly relates to extended release matrix compositions of carvedilol and one or more pharmaceutically acceptable excipients.

One embodiment of the present invention relates to extended release matrix composition and its dosage forms comprising carvedilol phosphate, polyethylene oxide, polyacrylic acid and one or more pharmaceutically acceptable excipients.

Another embodiment of this invention relates to extended release capsule dosage form suitable for once-daily administration comprising a plurality of mini-tablets comprising carvedilol phosphate having particle size d90 from 1 μm to 10 μm, polyethylene oxide, polyacrylic acid and one or more pharmaceutically acceptable excipients.

Yet another embodiment of the present invention provides the use of carvedilol phosphate compositions for the treatment of at least one of hypertension, heart failure and left ventricular dysfunction following myocardial infarction in a patient in need thereof.

### DETAILED DESCRIPTION OF THE INVENTION

The present invention relates to solid dosage forms comprising carvedilol phosphate and one or more pharmaceutically acceptable excipients.

The term “carvedilol” as used herein according to the present invention includes carvedilol in the form of free base or a pharmaceutically acceptable salt thereof preferably, carvedilol phosphate.

As used in this specification and the appended claims, the singular forms “a”, “an”, and “the” include plural references unless the context clearly dictates otherwise. Thus for example, reference to “a method” includes one or more methods, and/or steps of the type described herein and/or which will become apparent to those persons skilled in the art upon reading this disclosure so forth.

The term “excipient” means a pharmacologically inactive component such as a diluent, a binder, a disintegrant, a glidant, a lubricant, etc of a pharmaceutical product. The excipients that are useful in preparing a pharmaceutical composition are generally safe, non-toxic and are acceptable for human pharmaceutical use. Reference to an excipient includes both one and more than one such excipients.

By the term “solid dosage form” or “dosage form” or “composition” as used herein refers to a solid dosage form suitable for oral administration, such as a tablet, capsule, mini-tablets, spheroids, pellets, granules, pills and the like meant for extended release.

The term “extended release” used herein refer to a dosage form that provides gradual release of carvedilol phosphate over an extended period of time.

The term “poly acrylic acid” as used herein is synonymous to “carbomer” or “carbopol”.

One embodiment of the present invention relates to extended release matrix composition and its dosage forms comprising carvedilol phosphate, polyethylene oxide, polyacrylic acid and one or more pharmaceutically acceptable excipients.

Polyethylene oxide used according to the present invention is present in an amount of from about 5% w/w to about 50% w/w based on total weight of the composition, preferably from about 10% w/w to about 40% w/w.

Polyethylene oxide used according to the present invention has a molecular weight ranging from about 10,00,000 to about 70,00,000 daltons, preferably from 40,00,000 to 70,00,000 daltons. For example, polyox grades such as WSR N-12K, WSR N-60K, WSR-301, WSR coagulant and WSR-303 (commercially available from Dow Chemical Company) can be used.

Polyacrylic acid used according to the present invention is present in an amount of from about 1% w/w to about 10% w/w preferably, from about 3% w/w to about 7% w/w selected from one or more of polyacrylic acids having viscosity ranging from 4000 to 60000 cps in 0.5% w/v solution at pH 7.5 (as per USP32-NF27) preferably from 4000 to 40000 cps.

Polyacrylic acid used according to the present invention is a combination of low and high viscosity carbopols in a ratio of from 1:0.5 to 1:5 preferably from 1:1 to 1:3. For example, carbomer grades such as carbomer 934, carbopol 974P (carbomer 934P), carbopol 971P (carbomer 941), carbomer 1342 are useful in the present invention.

Polyethylene oxide and polyacrylic acid according to the present invention are present in a ratio of from 1:0.1 to 1:1, preferably from 1:0.125 to 1:0.5.

The composition according to present invention may further comprise excipients selected from one or more of diluents, binders, disintegrants, glidants and lubricants.

Diluents: Various useful diluents include but are not limited to microcrystalline cellulose, microtine cellulose, powdered cellulose, lactose anhydrous, lactose monohydrate, dibasic calcium phosphate, tribasic calcium phosphate, starch, pregelatinized starch, calcium carbonate, calcium sulfate, magnesium carbonate, magnesium oxide, dextrates, dextrin, dextrose, kaolin, maltodextrin, mannitol, xylitol and sorbitol and the like and combinations thereof.

Binders: Various useful binders include but are not limited to hydroxypropyl cellulose, low-substituted hydroxypropyl cellulose, hydroxypropyl methylcellulose, polyvinyl pyrrolidone, pregelatinized starch, powdered acacia, gelatin, guar gum, carbomers and the like and combinations thereof.

Disintegrants: Various useful disintegrants include but are not limited to sodium starch glycolate, croscarmellose sodium, crospovidone, and the like and combinations thereof. Preferred superdisintegrant is sodium starch glycolate.

Glidants: Various useful glidants include but are not limited to colloidal silicon dioxide, other forms of silicon dioxide, such as aggregated silicates and hydrated silica, magnesium silicate, magnesium trisilicate, talc, and the like and combinations thereof.

Lubricants: Various useful lubricants include but are not limited to talc, magnesium stearate, calcium stearate, zinc stearate, stearic acid, fumaric acid, palmitic acid, sodium stearyl fumarate, carnauba wax, hydrogenated vegetable oils, mineral oil, polyethylene glycols, and the like and combinations thereof.

Lubricants according to the present invention are used in an amount of from 0.5% w/w to 15% w/w based on total weight of the composition.

The composition according to the present invention is in the form of a tablet, mini-tablets, caplets, pellets, or a capsule, preferably mini-tablets.

Another embodiment of this invention relates to extended release capsule dosage form suitable for once-daily administration comprising a plurality of mini-tablets comprising carvedilol phosphate having particle size d90 from 1 μm to 10 μm, polyethylene oxide, polyacrylic acid and one or more pharmaceutically acceptable excipients.

Compositions of the present invention are prepared by direct compression technique comprising the steps of blending carvedilol phosphate, polymers and one or more other pharmaceutically acceptable excipients, compressing the blend to obtain tablets, preferably mini-tablets.

The mini-tablets according to the present invention have a diameter less than 5 mm and concavity less than 0.6 mm.

Alternatively the compositions of the present invention are prepared by wet granulation technique.

Wet granulation process comprise the steps of: (i) co-sifting and blending carvedilol phosphate along with desired intragranular excipients to form a dry mix, (ii) granulating the dry mix of step (i) using a suitable solvent(s) or a binder solution to form granules followed by drying, (iii) blending the granules of step (ii) with extragranular excipients, (iv) lubricating blend of step (iii) using suitable lubricant(s) and finally compressing the lubricated granules of step (iv) into tablets/mini-tablets of desired dimensions or filled into capsules.

The tablets or mini-tablets prepared according to the present invention are optionally coated with a film coating.

The plurality of mini-tablets prepared according to the present invention are preferably filled into capsules in an amount that shows efficacy therapeutically in a patient in need thereof.

Yet another embodiment of the present invention provides the use of carvedilol phosphate compositions for the treatment of at least one of hypertension, heart failure and left ventricular dysfunction following myocardial infarction in a patient in need thereof.

Certain specific aspects and embodiments of this invention are described in further detail by the examples below, which are provided only for the purpose of illustration and are not intended to limit the scope of the invention in any manner.

### EXAMPLES

### Example 1

### Solid Dosage Forms of Carvedilol Phosphate:

| Ingredient | Example 1(% w/w) |
|---|---|
| Carvedilol phosphate | 23.58 |
| Lactose monohydrate | 49.69 |
| Polyethylene oxide | 19.44 |
| Carbopol 971P | 1.67 |
| Carbopol 974P | 2.50 |
| Colloidal silicon dioxide | 1.11 |
| Lubrication | |
| Magnesium stearate | 2.00 |
| Total | 100 |

### Manufacturing Process:

1. Carvedilol phosphate, carbopol 974P, carbopol 971P were co-sifted through mesh #40,
2. material of step 1 was sifted through mesh #40 with lactose monohydrate, polyethylene oxide and colloidal silicon dioxide and blended for 10 minutes,
3. magnesium stearate was sifted through mesh #60,
4. blend of step 2 was lubricated with magnesium stearate of step 3,
5. lubricated blend of step 4 was compressed into mini-tablets,
6. mini-tablets of step 5 were filled into capsules equivalent to label claim.

### Study on Dissolution Time:

Comparative dissolution profile was established between marketed Coreg CR capsules and capsules of the present invention with the following attributes.

Dissolution medium: 0.1 N HCl

### Volume: 900 ml

### Apparatus: USP II (Paddle)

### Speed: 100 rpm

% of drug release at different time intervals

| Time (in hours) | Coreg CR ® | Example 1 |
|---|---|---|
| 1 | 11 | 9 |
| 4 | 35 | 24 |
| 8 | 60 | 56 |
| 12 | 75 | 78 |
| 18 | 85 | 92 |
| 24 | 91 | 97 |

### Example 2 to 3

### Solid Dosage Forms of Carvedilol Phosphate:

| | Example 2 | Example 3 |
|---|---|---|
| Ingredient | (mg/capsule) | (mg/capsule) |
| Carvedilol phosphate | 42.45 | 42.45 |
| Lactose monohydrate | 94.55 | 73.95 |
| Polyethylene oxide | 32.00 | 40.00 |
| Carbopol 971P | 2.70 | 2.00 |
| Carbopol 974P | 2.70 | 6.00 |
| Colloidal silicon dioxide | 2.00 | 2.00 |
| Lubrication | | |
| Magnesium stearate | 3.60 | 3.60 |
| Total | 180 | 170 |

### Manufacturing Process:

1. Carvedilol phosphate, carbopol 974P, carbopol 971P were co-sifted through mesh #40,
2. material of step 1 was sifted through mesh #40 with lactose monohydrate, polyethylene oxide and colloidal silicon dioxide and blended for 10 minutes,
3. magnesium stearate was sifted through mesh #60,
4. blend of step 2 was lubricated with magnesium stearate of step 3,
5. lubricated blend of step 4 was compressed into mini-tablets,
6. mini-tablets of step 5 were filled into capsules equivalent to label claim.

### Example 4 to 6

### Solid Dosage Forms of Carvedilol Phosphate:

| | Example 4 | Example 5 | Example 6 |
|---|---|---|---|
| Ingredient | (% w/w) | (% w/w) | (% w/w ) |
| Carvedilol phosphate | 23.58 | 23.58 | 23.58 |
| Lactose monohydrate | 49.53 | 49.53 | 44.14 |
| Polyethylene oxide | 19.44 | 19.44 | 25.00 |
| Carbopol 971P | 1.67 | 0.83 | 1.67 |
| Carbopol 974P | 1.67 | 2.50 | 2.50 |
| Colloidal silicon dioxide | 1.11 | 1.11 | 1.11 |
| Lubrication | | | |
| Magnesium stearate | 3.00 | 3.00 | 2.00 |
| Total | 100 | 100 | 100 |

### Manufacturing Process:

1. Carvedilol phosphate, carbopol 974P, carbopol 971P were co-sifted through mesh #40,
2. material of step 1 was sifted through mesh #40 with lactose monohydrate, polyethylene oxide and colloidal silicon dioxide and blended for 10 minutes,
3. magnesium stearate was sifted through mesh #60,
4. blend of step 2 was lubricated with magnesium stearate of step 3,
5. lubricated blend of step 4 was compressed into mini-tablets,
6. mini-tablets of step 5 were filled into capsules equivalent to label claim.

### Example 7 to 9

### Solid Dosage Forms of Carvedilol Phosphate:

| | Example 7 | Example 8 | Example 9 |
|---|---|---|---|
| Ingredient | (% w/w) | (% w/w) | (% w/w) |
| Carvedilol phosphate | 23.58 | 23.58 | 23.58 |
| Lactose monohydrate | 49.53 | 49.53 | 44.14 |
| Polyethylene oxide | 19.44 | 19.44 | 25.00 |
| Carbomer 1342 | 1.67 | 0.83 | 1.67 |
| Carbomer 934 | 1.67 | 2.50 | 2.50 |
| Colloidal silicon dioxide | 1.11 | 1.11 | 1.11 |
| Lubrication | | | |
| Magnesium stearate | | | |
| Total | 100 | 100 | 100 |

### Manufacturing Process:

1. Carvedilol phosphate, carbomer 1342, carbopol 934 were co-sifted through mesh #40,
2. material of step 1 was sifted through mesh #40 with lactose monohydrate, polyethylene oxide and colloidal silicon dioxide and blended for 10 minutes,
3. magnesium stearate was sifted through mesh #60,
4. blend of step 2 was lubricated with magnesium stearate of step 3,
5. lubricated blend of step 4 was compressed into mini-tablets,
6. mini-tablets of step 5 were filled into capsules equivalent to label claim.

### Example 10 to 14

### Solid Dosage Forms of Carvedilol Phosphate:

| | Example | Example | Example | Example | Example |
|---|---|---|---|---|---|
| | 10 | 11 | 12 | 13 | 14 |
| Ingredient | (% w/w) | (% w/w) | (% w/w) | (% w/w) | (% w/w) |
| Carvedilol phosphate | 23.49 | 23.49 | 23.49 | 23.49 | 23.49 |
| Lactose monohydrate | 36.24 | 26.24 | 30.24 | 34.24 | 36.24 |
| Polyethylene oxide | 31.94 | 31.94 | 31.94 | 31.94 | 31.94 |
| Carbopol 971P | 2.08 | 2.08 | 2.08 | 2.08 | 2.08 |
| Carbopol 974P | 3.13 | 3.13 | 3.13 | 3.13 | 3.13 |
| Colloidal silicon dioxide | 1.11 | 1.11 | 1.11 | 1.11 | 1.11 |
| Lubrication | | | | | |
| Magnesium stearate | 2.00 | — | — | — | 1.00 |
| Talc | — | 12.00 | — | — | 1.00 |
| Zinc stearate | — | — | 8.00 | — | — |
| Calcium stearate | — | — | — | 4.00 | — |
| Total | 100 | 100 | 100 | 100 | 100 |

### Manufacturing Process:

1. Carvedilol phosphate, carbopol 974P, carbopol 971P were co-sifted through mesh #40,
2. material of step 1 was sifted through mesh #40 with lactose monohydrate, polyethylene oxide and colloidal silicon dioxide and blended for 10 minutes,
3. selected lubricant(s) was sifted through mesh #60,
4. blend of step 2 was lubricated with lubricant of step 3,
5. lubricated blend of step 4 was compressed into mini-tablets,
6. mini-tablets of step 5 were filled into capsules equivalent to label claim.

### Example 15 to 17

### Solid Dosage Forms of Carvedilol Phosphate:

| | Example 15 | Example 16 | Example 17 |
|---|---|---|---|
| Ingredient | (% w/w) | (% w/w) | (% w/w) |
| Carvedilol phosphate | 23.49 | 23.49 | 23.49 |
| Lactose monohydrate | 32.24 | 32.24 | 49.78 |
| Polyethylene oxide | 31.94 | 31.94 | 19.44 |
| Carbopol 971P | 2.08 | 2.08 | 1.67 |
| Carbopol 974P | 3.13 | 3.13 | 2.50 |
| Colloidal silicon dioxide | 1.11 | 1.11 | 1.11 |
| Purified water | qs | — | — |
| Isopropyl alcohol | — | qs | qs |
| Lubrication | | | |
| Talc | 6.00 | 6.00 | 6.00 |
| Total | 100 | 100 | 100 |

### Manufacturing Process:

1. Carvedilol phosphate, lactose monohydrate, carbopol 974P, carbopol 971P were co-sifted through mesh #40,
2. material of step 1 was dry mixed and granulated using purified water/isopropyl alcohol,
3. the wet mass was dried and the granules so obtained were passed through mesh #40,
4. polyethylene oxide was sifted through mesh #30, colloidal silicon dioxide was sifted through mesh #40, mixed to granules of step 3 and blended,
5. talc was sifted through mesh #60 and blend of step 4 was lubricated with talc,
6. lubricated blend of step 5 was compressed into mini-tablets,
7. mini-tablets of step 6 were filled into capsules equivalent to label claim.

1. An extended release matrix composition comprising i) carvedilol phosphate, ii) polyethylene oxide, iii) polyacrylic acid and iv) one or more pharmaceutically acceptable excipients.

2. The composition of claim 1, wherein the polyethylene oxide has a molecular weight of about 10,00,000 to about 70,00,000.

3. The composition of claim 1, wherein the polyacrylic acid is a combination of polyacrylic acids having a viscosity ranging from 4000 to 60000 cps in 0.5% w/v solution.

4. The composition of to claim 1, wherein the polyethylene oxide and the polyacrylic acid are present in a ratio of from 1:0.1 to 1:1, preferably from 1:0.125 to 1:0.5.

5. The composition of claim 1, wherein the excipients are selected from one or more of diluents, binders, disintegrants, glidants and lubricants.

6. The composition of claim 5, wherein the lubricants are one or more of magnesium stearate, talc, zinc stearate, calcium stearate, sodium stearyl fumarate and stearic acid in an amount of from 0.5% w/w to 15% w/w based on the total weight of the composition.

7. The composition of claim 1, in the form of a tablet, mini tablets, caplets, pellets, or a capsule.

8. An extended release capsule dosage form suitable for once-daily administration comprising a plurality of mini-tablets, the mini-tablets comprising carvedilol phosphate having a particle size d90 from 1 μm to 10 μm, polyethylene oxide, polyacrylic acid, and one or more pharmaceutically acceptable excipients.

9. The extended release capsule dosage form of claim 8, wherein the mini-tablets have a diameter less than 5 mm, a concavity less than 0.6 mm, and the mini-tablets are filled into a capsule.

10. The extended release capsule dosage form of claim 8, prepared by a direct compression technique comprising the steps of:
i) blending carvedilol phosphate, polyethylene oxide, polyacrylic acid, and one or more pharmaceutically acceptable excipients to form a blend,
ii) compressing the blend of step (i) to obtain mini-tablets,
iii) optionally coating the mini-tablets of step (ii), and
iv) filling the mini-tablets of step (iii) into capsules.

11. The extended release capsule dosage form of claim 8, prepared by a wet granulation technique comprising the steps of:
i) granulating carvedilol phosphate, polyethylene oxide, polyacrylic acid, and one or more pharmaceutically acceptable excipients to form granules,
ii) compressing the granules of step (i) to obtain mini-tablets,
iii) optionally coating the mini-tablets of step (ii),
iv) filling the mini-tablets of step (iii) into capsules.

12. A method of treating heart failure, left ventricular dysfunction following myocardial infarction and hypertension in a patient in need thereof, comprising administering to the patient the composition of claim 1.
